(19) United States
(12) Patent Application Publication
(10) Pub. No.: US 2004/0186314 A1
(43) Pub. Date: Sep. 23, 2004

(54) Process for the preparation of enantiomerically enriched n-acyl-beta-amino acid derivatives by enantioselective hydrogenation of the corresponding (z)-enamides

(76) Inventors: Christophe Guillaume Malan, Basel (CH); Christopher Cobley, Cambridge (GB)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/485,188

(22) PCT Filed: Aug. 16, 2002
(86) PCT No.: PCT/US02/26142

(30) Foreign Application Priority Data
Aug. 17, 2001 (GB) 0120167.2
Aug. 17, 2001 (GB) 120167.2

(51) Int. Cl. C07C 235/04

(52) U.S. Cl. 560/155

(57) ABSTRACT

A process for the preparation of an enantiomerically enriched beta-amino acid derivative of formula (1), or the opposite enantiomer thereof, wherein R1 is an optionally substituted alkyl, aryl or heteroaryl group of up to 20 C atoms, R2 is ann alkyl group of up to 20 C atoms, and R3 is H or an alkyl or aryl group of up to 20 C atoms, which comprises asymmetric hydrogenation of the (z)-enamide precursor (2) in an alcohol solvent or cosolvent, catalysed by a cationic rhodium complex of a chiral phosphine ligand having the partial formula (3), wherein n is 0 to 6 and R represents at least one non-hydrogen organic group of up to 20 C atoms.

This invention relates to processes suitable for the large-scale preparation of enantiomerically enriched N-acyl β-amino acid derivatives. In particular, it relates to enantioselective hydrogenation of the corresponding (Z)-enamide precursors.

Optically active β-amino acids are found in many natural products, such as β-peptides (Gellman, Acc. Chem. Res., 1998, 31, 160 and references cited therein). These and other compounds derived from β-amino acid building blocks have a wide spectrum of biomedical applications and therefore β-amino acids have become important commercial targets.

Numerous methods for the synthesis of β-amino acids in enantiopure or enantioenriched form have been devised [for an overview, see: E. Juaristi, Ed., Enantioselective Synthesis of β-Amino Acids, Wiley-VCH: New-York (1997)], but most of them have significant drawbacks, limiting commercial utility. For example, enzymatic resolution entails the obvious net Weight loss of at least 50 percent of material. Widely used methods based on stoichiometric chiral auxiliaries give reliable results for laboratory scale work, but because of the mass contribution of the chiral auxiliary, atom economy is poor and is thus a limitation for large-scale applications.

A more attractive commercial approach to β-amino acids is through homogeneous asymmetric hydrogenation, which offers the advantages of catalyst fine-tuning through rapid screening followed by clean, atom-economical processes and recognised ease of scale-up. Suitable precursors are β-(acylamino)acrylate esters (i), as either (E)- or (Z)-isomers, in which the N-acyl group serves as a binding site for transition metal-based catalysts containing chiral ligands. These substrates are easily prepared through standard protocols, most conveniently from the corresponding β-keto ester by reaction of with ammonia followed by N-acylation (Zhu, Chen and Zhang, J. Org. Chem., 1999, 64, 6907) or more directly by reaction with acetamide (Overden, Capon, Lacey, Gill, Friedel and Wadsworth, J. Org. Chem., 1999, 64, 1140). These methods tend to give geometric mixtures of (i) in which the (Z)-isomer is the major component; in some cases, pure (Z)-i is obtained.

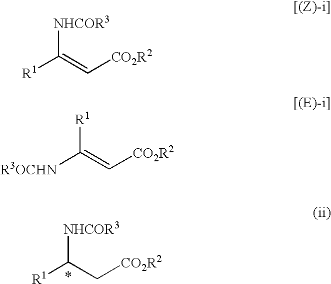

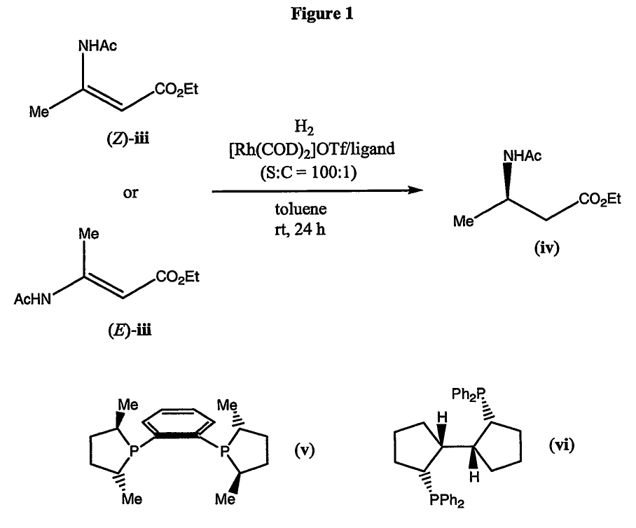

It is reported in the literature that rhodium and ruthenium complexes of chiral diphosphine ligands are effective catalysts for the asymmetric hydrogenation of β-(acylamino)acrylate esters (i), in which R¹ is alkyl or aryl, R² and R³ are alkyl. A limitation of ruthenium catalysis (Lubell, Kitamura and Noyori, Tetrahedron: Asymmetry, 1991, 2, 543) is that hydrogenation of (E)- and (Z)-i with the same catalyst leads to β-amino acid derivatives (ii) of opposite absolute configuration. Moreover, whilst pure (E)-i could be quantitatively hydrogenated with high enantioselectivity (87-96 percent ee) in the presence of a ruthenium BINAP complex, hydrogenation of (Z)-i proceeded in only 5-9 percent ee. Thus, ruthenium catalysis is only a practical method for the more scarce (E)-β-(acylamino)acrylate esters (E)-i. In contrast, rhodium complexes of chiral diphosphine methods can facilitate. the enantioconvergent hydrogenation of (E)- and (Z)-i, although significant differences in the reactivity and/or enantioselectivity, in favour of (E)-i are usually observed (Zhu, Chen and Zhang, J. Org. Chem., 1999, 64, 6907). FIG. 1/Table 1 shows illustrative results, reported for the hydrogenation of ethyl 3-acetamido-2-butenoate (iii), with toluene as solvent, catalysed by rhodium complexes of (R,R)-methyl DuPHOS (v) and (R,R)-BICP (vi). With both catalysts (Entries 1 and 2), hydrogenation of (E)-iii proceeded quantitatively at 40 psi to give highly enantioenriched (R)-ethyl 3-acetamidobutanoate (iv). In comparison, hydrogenation of (Z)-iii (Entries 3 and 4) required a hydrogen pressure of 294 psi to proceed to completion over 24 hours, at a substrate:catalyst ratio of 100:1, and enantioselectivity was reduced. In particular, the results in Entry 4 suggest that DuPHOS-based catalysts have insufficient selectivity for the hydrogenation of (Z)-β-(acylamino)acrylate esters.

TABLE 1
Asymmetric hydrogenation of ethyl 3-
acetamido-2-butenoate (iii)

| | | | | | Percent |
|---|---|---|---|---|---|
| Entry | Substrate | Ligand | H₂ pressure | Percent ee | Conversion |
| 1 | (E)-iii | vi | 40 psi | 96.0 | 100 |
| 2 | (E)-iii | v | 40 psi | 98.7 | 100 |
| 3 | (Z)-iii | vi | 294 psi | 88.0 | 100 |
| 4 | (Z)-iii | v | 294 psi | 62.3 | 100 |

It is evident from the prior literature that there is scope for improvement in the asymmetric hydrogenation of (Z)-β-(acylamino)acrylate esters, in order to establish industrially viable processes conducted at high substrate:catalyst ratios (typically of at least 1000:1). This factor is paramount with rhodium-based catalyst due to the high cost of the metal. For optimum process economics, a method allowing rapid and selective hydrogenation of (Z)/(E)-geometric mixtures of β-(acylamino)acrylate esters is also sought.

For related studies published after the original priority date of this patent application, see Heller, Holz, Drexler, Lang, Drauz, Krimmer and Börner, J. Org. Chem., 2001, 66, 6816; Lee and Zhang, Org Lett., 2002, 4, 2429.

This invention is based on the discovery that the use of alcohol solvents enables the rapid and highly enantioselective catalytic hydrogenation of the (Z)-enamide precursors of N-acyl β-amino acid derivatives, catalysed by cationic rhodium complexes of chiral phosphine ligands. This simple expedient provides the basis of an industrially useful and atom-efficient process to these products.

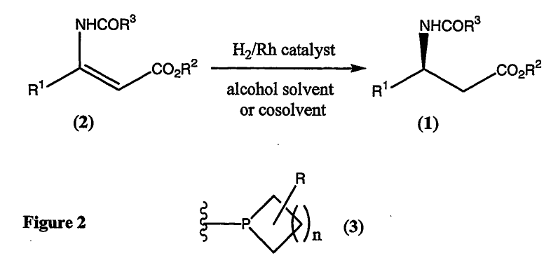

The present invention encompasses a novel process as depicted in FIG. 2, and provides an effective means of accessing an enantiomerically enriched β-amino acid derivative of formula (1), or the opposite enantiomer thereof, from the (Z)-enamide precursor (2). In the product (1) and the substrate (2), R¹ is an optionally substituted alkyl, aryl or heteroaryl group of up to 20 C atoms, R² is an alkyl group of up to 20 C atoms, and R³ is H or an alkyl or aryl group of up to 20 C atoms. The process comprises asymmetric hydrogenation of the substrate (2) and is distinguished from the prior art by the use of an alcohol solvent or cosolvent in combination with, as catalyst, a cationic rhodium complex of a chiral phosphine ligand having the partial formula (3), wherein n is 0 to 6 and R represents at least one non-hydrogen organic group of up to 20 C atoms. Where two or more R groups are present, such R groups may be the same or different, may optionally be joined to form a ring, and may optionally obtain heteroatoms. Ligands having such structural variations will be readily identifiable by those skilled in the art.

In the process of the present invention, the (Z)-enamide precursor (2) may either be used in geometrically pure form or in admixture with (E)-enamide precursor (4). In the latter variant, (4) also undergoes hydrogenation to afford (1), enriched in the same enantiomer as obtainable from (2) and usually at a rate comparable to or faster than (2). Combined with the prior step of synthesis of the (E)/(Z)-enamide mixture, for example by amination-acylation of the corresponding β-keto ester, this can provide a highly atom-efficient route to the desired product (1).

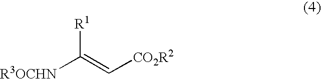

In preferred embodiments of the present invention, R¹, R² and R³ are each independently alkyl and are more preferably methyl or n-alkyl, R² typically being methyl or ethyl. The alcohol solvent or cosolvent is typically selected from the group comprising methanol, ethanol, isopropanol and triflouroethanol. The moiety R²OH and the alcohol can be the same, so as to preclude the side reaction of ester exchange. However, this is not always necessary, for example trifluoroethanol can be an effective solvent for substrates (2) in which R² is methyl or ethyl.

In the catalyst used to effect the process, the chiral phosphine ligand is usually of formula (5) or (6), wherein Linker and R′ are independently any non-hydrogen organic or organometallic group of up to 30 C atoms. Preferably, the ligand is a bidentate ligand of formula (6) and n is 2; more preferably, the ligand is of formula (7), or an opposite enantiomer thereof, the optimum group R being dependent on the particular hydrogenation substrate, and readily determined by screening a series of catalysts. R is a non-hydrogen organic group of up to 20 C atoms and is usually is a C₁₋₈ linear or branched alkyl group.

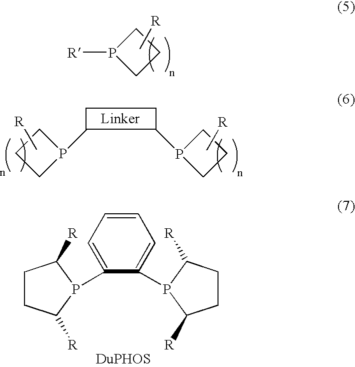

The cationic rhodium complex used as catalyst is usually of the form [Rh(6)(COD)]⁺BF₄, although it will be recognised by those skilled in the art that COD (1,5-cyclooctadiene) may be replaced by an alternative diene such as NBD (2,5-norbornadiene), and that BF₄⁻may be replaced by an alternative counterion such as PF₆⁻. In this context, the term “catalyst” refers to the isolated pre-catalyst that is added to the reaction vessel for hydrogenation and which typically undergoes a change in composition in situ to generate one or more catalytically active species. Equivalent catalysis may be achieved by generation of catalytically active species from the chiral ligand (6) and an achiral rhodium-containing precursor.

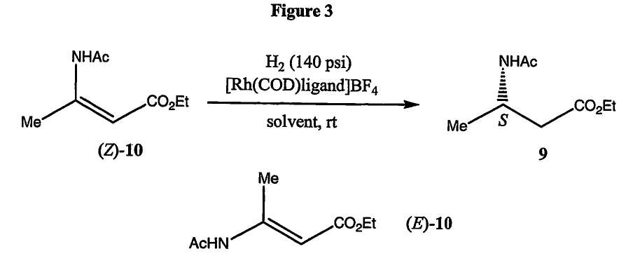

In one embodiment of the present invention, ethyl 3-acetamido-2-butanoate was obtained through the rapid and highly enantioselective asymmetric hydrogenation of (Z)-ethyl 3-acetamido-2-butenoate [(Z)-10] under carefully selected conditions (FIG. 3). Results of comparative experiments are summarised in Table 2. Entry 1 (Rh-MeDuPHOS catalyst and toluene solvent) shows poor substrate conversion at high substrate:catalyst ratio and moderate enantioselectivity. Entry 2 shows the marked effect of changing the solvent to methanol, the same reaction proceeding to completion in less than 1 minute. Having achieved high reactivity and conversion, Entries 3 & 4 show incremental improvements in enantioselectivity through use of homologous catalysts containing EtDuPHOS and ⁱPrDuPHOS ligands. With the latter, an effective process is also achieved at a higher substrate:catalyst ratio (Entry 5). Entries 6-9 show further experiments with the preferred Rh-ⁱPrDuPHOS catalyst and variation of the alcohol solvent. Trifluoroethanol (TFE) gives the best process in terms of both rate and enantioselectivity (Entry 8) and is also highly effective when used as a cosolvent with methanol (Entry 9). Entries 10-12 show highly effective enantioconvergent hydrogenation of a 1:1 mixture of (Z)-10 and (E)-10. Entry 13 is a control experiment showing rapid and highly selective asymmetric hydrogenation of pure (E)-10 in TFE.

TABLE 2
Asymmetric hydrogenation of ethyl 3-acetamido-2-butenoate (10)

| | | | | | Reaction | | Percent | Percent |
|---|---|---|---|---|---|---|---|---|
| Entry | Substrate | Ligant | Solvent | S:C | Time | | ee | conversion |
| 1 | (Z)-10 | (R,R)-Me DuPHOS | toluene | 100:1 | 4 | h | 79.7* | 6.5 |
| 2 | (Z)-10 | (R,R)-Me DuPHOS | MeOH | 100:1 | <1 | min | 58.0* | 100 |
| 3 | (Z)-10 | (S,S)-Et DuPHOS | MeOH | 100:1 | 1.5 | min | 79.6 | 100 |
| 4 | (Z)-10 | (R,R)-ⁱPr DuPHOS | MeOH | 100:1 | 1.5 | min | 90.0 | 100 |
| 5 | (Z)-10 | (R,R)-ⁱPr DuPHOS | MeOH | 1000:1 | 1.5 | h | 87.0 | 100 |
| 6 | (Z)-10 | (R,R)-ⁱPr DuPHOS | EtOH | 100:1 | 30 | min | 85.6 | 94 |
| 7 | (Z)-10 | (R,R)-ⁱPr DuPHOS | ⁱPrOH | 100:1 | 30 | min | 82.8 | 92 |
| 8 | (Z)-10 | (R,R)-ⁱPr DuPHOS | TFE | 100:1 | <2 | min | 92.2 | 100 |
| 9 | (Z)-10 | (R,R)-ⁱPr DuPHOS | MeOH/TFE | 1000:1 | <5 | min | 91.8 | 100 |
| | | | (4/1) | | | | | |
| 10 | (Z)/(E)-10 | (R,R)-ⁱPr DuPHOS | MeOH | 100:1 | 10 | min | 94.0 | 100 |
| | (1/1) | | | | | | | |
| 11 | (Z)/(E)-10 | (R,R)-ⁱPr DuPHOS | MeOH | 1000:1 | 2 | h | 92.0 | 100 |
| | (1/1) | | | | | | | |
| 12 | (Z)/(E)-10 | (R,R)-ⁱPr DuPHOS | TFE | 1000:1 | 40 | min | 95.3 | 100 |
| | (1/1) | | | | | | | |
| 13 | (E)-10 | (R,R)-ⁱPr DuPHOS | TFE | 100:1 | <2 | min | 100 | 99.2 |

*product enriched in (R)-enantiomer

In another embodiment of the present invention, [(ⁱPrDuPHOS)Rh(COD)]BF₄ was shown to be an effective catalyst for the asymmetric hydrogenation of (Z)-ethyl 3-acetamido-2-phenyl-2-propenoate, to produce ethyl 3-acetamido-2-phenylpropanoate in 77 percent ee (at 140 psi H₂ pressure), increasing to 89 percent ee at 30 psi H₂.

In summary, the process of the present invention provides an effective means of preparing a wide range of enantiomerically enriched N-acyl β-amino acid derivatives. In order to be economically viable, it is important that enantiomeric enrichment of the product obtained by asymmetric hydrogenation is at least 70 percent ee, and is preferably at least 90 percent ee, or higher. If necessary, any shortfall in ee can subsequently be corrected by standard techniques such as selective enzyme-catalysed hydrolysis of the ester or amide group of the unwanted enantiomer of the hydrogenation product.

The present invention is further illustrated by the following examples.

### EXAMPLE 1

Preparation of (Z)- and (E)-ethyl 3-acetamido-2-butenoate

The title compounds were prepared by conventional acetylation (Py, DCM, AcCl, DMAP catalyst) of commercially available ethyl 3-amino-2-butenoate (E:Z mixture). The crude material obtained by extractive work-up was purified by crystallisation from diethyl ether. The (E)-isomer crystallized first (isolated yield 30-35 percent; unoptimised), followed by the (Z)-isomer (isolated yield 20 percent, unoptimised). ¹H and ¹³C-NMR spectra were consistent with reported data (Zhu, Chen and Zhang, J. Org. Chem., 1999, 64, 6907).

### EXAMPLE 2

Representative Procedure for Small-scale Hydrogenation of (Z)- and (E)-ethyl 3-acetamido-2-butenoate (re. Table 2)

In a glass liner, the precatalyst (0.01 mmol) and the starting material (171 mg, 1.0 mmol) were weighed. A stirring cross was added and the liner was placed in a 50 mL autoclave. The autoclave was purged 4 times with 120-160 psi N₂ and 7-8 times with H₂ (same pressure). Methanol (3 mL; reagent grade) was added and the autoclave purged with 140 psi H₂, at least 8 times. Vigorous stirring was started. When hydrogen uptake had ceased (7-8 psi), pressure was released and an aliquot immediately taken, diluted with diethyl ether or ethyl acetate and analysed by chiral GC on a Chirasil Dex-CB column.

### EXAMPLE 3

Hydrogenation of a 1:1 Mixture of (Z)- and (E)-ethyl 3-acetamido-2-butenoate

In a glass liner, the [Rh(S,S)-i-Pr-DuPHOS(COD)]BF₄ pre-catalyst (2.1 mg, 0.0029 mmol) and the starting material (255 mg of each isomer, overall 3.0 mmol) were weighed. A stirring cross was added and the liner was placed in a 50 mL autoclave. It was purged 4 times with 120-160 psi N₂, 7-8 times with H2 (same pressure). Trifluoroethanol (9 mL; reagent grade) was added and the autoclave purged with 140 psi H₂, at least 8 times. Vigorous stirring was started. When hydrogen uptake had ceased (24 psi, 40 min), pressure was released and an aliquot immediately taken, and diluted with diethyl ether. Analysis by chiral GC: 100 percent conversion of substrate to (S)-ethyl 3-acetamidobutanoate of 95.3 percent ee.

### EXAMPLE 4

Preparation of (Z)-ethyl 3-acetamido-3-phenyl-2-propenoate

Ammonium acetate (21.1 g, 0.273 mol) was added to a solution of ethyl benzoylacetate (35.0 g, 0.182 mol) in ethanol (200 ml) and the mixture refluxed for 18 h. After removal of all volatiles in vacuo, the residue was suspended in CHCl₃ (200 ml). After filtration of the resulting solid and washing with CHCl₃ (2×100 ml), the combined filtrate was washed with water (150 ml) and brine (150 ml) then dried over magnesium sulphate. Removal of all volatiles on a rotary evaporator gave crude ethyl 3-amino-3-phenyl-2-propenoateas a yellow oil (94 percent). This was subsequently used without further purification. A pyridine solution (30ml) of crude β-amino ester (6.71 g, 0.035 mol) was cooled to −78° C. and acetyl chloride (2.5 ml, 0.036 mol) added dropwise over 30 min. Once addition was complete, the reaction mixture was allowed to warm to room temperature and left stirring for 16 h, during which time a white precipitate appeared. The reaction mixture was diluted with MTBE (30 ml) and the solution washed with HCl (1.0 M) until the aqueous phase remained acidic (4×40 ml). The organic phase was separated, washed with NaHCO₃ (saturated, 50 ml) and brine (50 ml), and dried over magnesium sulphate. After filtration, all volatiles were removed under reduced pressure. Chromatography of the reaction residue on silica with EtOAc in heptane (1:3) as eluant gave the (Z) isomer followed by a by-product and then the (E) isomer. The (E) isomer crystallised upon standing while the (Z) isomer remained an oil.

### EXAMPLE 5

Asymmetric Hydrogenation of (Z)-ethyl 3-acetamido-3-phenyl-2-propenoate

A glass liner was charged with [Rh(R,R)i-Pr-DuPHOS(COD)]BF₄ pre-catalyst (3.0 mg, 0.005 mmol) and a magnetic stirrer bar. The liner was placed in the base of a 50 ml Parr pressure vessel and the reactor assembled, flushed with nitrogen, and then purged with hydrogen (5 pressurisation/release cycles, 100 psi). A MeOH (5 ml) solution of (Z)-ethyl 3-acetamido-2-phenyl-2-propenoate (117 mg, 0.5 mmol) was added through the septum of the reactor via syringe. The reactor was purged once more with hydrogen (3 pressure/release cycles, 10 psi). The reactor was finally pressurised to 140 psi with hydrogen and stirred at room temperature for 2.5 h. The hydrogen was then released and the solution in the glass liner transferred to a vial. A sample was prepared and submitted for conversion and e.e. analysis by ¹H NMR and SFC electrophoresis (>98 percent conv., 77 percent e.e.). In a comparative experiment at 30 psi H₂ the same product was obtained in 89 percent ee.

What is claimed is:

1. A process for the preparation of an enantiomerically enriched β-amino acid derivative of formula (1), or the opposite enantiomer thereof, wherein R¹ is an optionally substituted alkyl, aryl or heteroaryl group of up to 20 C atoms, R² is an alkyl group of up to 20 C atoms, and R³ is H or an alkyl or aryl group of up to 20 C atoms, which comprises asymmetric hydrogenation of the (Z)-enamide precursor (2) in an alcohol solvent or cosolvent, catalysed by a cationic rhodium complex of a chiral phosphine ligand having the partial formula (3), wherein n is 0 to 6 and R represents at least one non-hydrogen organic group of up to 20 C atoms.

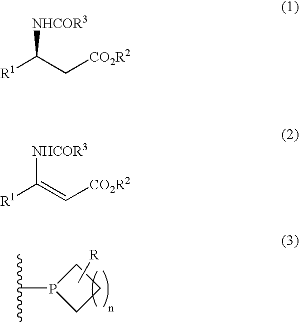

2. A process according to claim 1, wherein the (Z)-enamide precursor (2) is geometrically pure.

3. A process according to claim 1, wherein the (Z)-enamide precursor (2) is in admixture with (E)-enamide (4) and the latter is also undergoes hydrogenation.

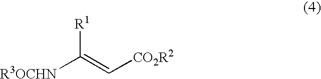

4. A process according to any preceding claim, wherein R¹ is alkyl.

5. A process according to claim 4, wherein R¹ is methyl or n-alkyl.

6. A process according to any preceding claim, wherein R¹ is aryl.

7. A process according to any preceding claim, wherein R² is alkyl.

8. A process according to claim 7, wherein R² is methyl or ethyl.

9. A process according to any preceding claim, wherein R³ is alkyl.

10. A process according to claim 9 wherein R³ is methyl or n-alkyl.

11. A process according to any preceding claim wherein the alcohol solvent or cosolvent is selected from the group comprising methanol, ethanol, isopropanol and trifluoroethanol.

12. A process according to claim 11 wherein the moiety R²OH and the alcohol are the same.

13. A process according to claim 12 wherein the moiety R²OH and the alcohol are different.

14. A process according to any preceding claim, wherein the chiral phosphine ligand is of formula (5) or (6), wherein Linker and R′ are independently any non-hydrogen organic or organometallic group of up to 30 C atoms.

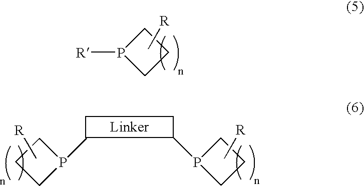

15. A process according to claim 14, wherein the ligand is of formula (6) and n is 2.

16. A process according to claim 15, wherein the ligand is of formula (7), or an opposite enantiomer thereof.

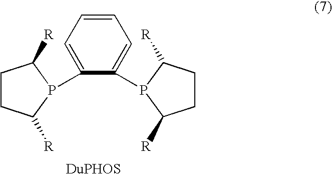

17. A process according to claim 16, wherein R in (7) is a C₁₋₈ linear or branched alkyl group.

18. A process according to claim 17, wherein R is isopropyl.

19. A process according to claim 14 or claim 15, wherein the ligand is incorporated into a precatalyst of formula [Rh(6)(diene)]⁺X⁻, wherein the diene is either COD or NBD and X⁻ is selected from the group comprising BF₄⁻, PF₆⁻, TfO⁻, tetra[3,5-bis(trifluromethyl)phenyl]borate and halide.

20. A process according to claim 19 wherein the diene is COD and X⁻ is BF₄⁻.

21. A process according to any preceding claim, giving β-aimino acid derivative (1) in at least 70 percent ee.

22. A process according to claim 21, giving β-amino acid derivative (1) in at least 90 percent ee.
